(19) United States
(12) Patent Application Publication
(10) Pub. No.: US 2022/0346883 A1
(43) Pub. Date: Nov. 3, 2022

(54) SYSTEMS AND METHODS FOR TARGETING A FEATURE ON A SURGICAL DEVICE

(71) Applicant: Mason James BETTENGA, Memphis, TN (US)

(72) Inventor: Mason James BETTENGA, Memphis, TN (US)

(21) Appl. No.: 17/732,273

(22) Filed: Apr. 28, 2022

Related U.S. Application Data
(60) Provisional application No. 63/182,087, filed on Apr. 30, 2021.

(51) Int. Cl. A61B 34/20; G01D 5/353; G01L 1/24

(52) U.S. Cl. CPC A61B 34/20; G01D 5/35316; G01L 1/246

(57) ABSTRACT

A system and method for targeting a feature on a surgical device, includes a shape sensing element coupled to a surgical device. A guide system having a moveable guide aperture is coupled to the surgical device in communication with the shape sensing element. An interrogator is operable to poll the shape sensing element for information related to the deflection of the targeted feature coupled in communication with a portion of the shape sensing element. A data processor is operable to communicate with the interrogator and provide adjustment information to the user related to the change in shape of the shape sensing element, which is related to a translation of the guide aperture with respect to the first device end such that the guide axis is aligned with the target axis. The shape sensing element may comprise at least one optical fiber, which may comprise a set of Bragg Gratings.

### CROSS REFERENCE TO RELATED APPLICATIONS

This application claims priority to U.S. Provisional App. No. 62/966,576 filed Apr. 30, 2021, titled SYSTEMS AND METHODS FOR TARGETING A FEATURE ON A NON-RIGID SURGICAL DEVICE, herein incorporated by reference in its entirety for all purposes.

### FIELD OF THE INVENTION

The present invention relates to devices and methods for orthopedic tissue reconstruction procedures requiring the alignment of a surgical tool to a hidden feature on a surgical device.

### BACKGROUND

In the field of orthopedic surgery, intramedullary rods, or ‘nails’, are a common means of providing stable, weight-bearing fixation during the healing period of a fractured bone. The intramedullary space is prepared in a reaming process, and a rod is introduced either from the proximal end of the bone or the distal, depending on the particular anatomy and pathology. Intramedullary nail introduction occurs through an entrance hole formed in a first segment of the fractured bone and is then advanced through the prepared canal, crossing the fracture line, and subsequently advanced through the canal of a second bone segment. Bi-cortical fixation of both the proximal and distal segments of the bone to the ends of the nail is accomplished by drilling through both cortical walls of the bone to form a hole colinear with a hole transverse to the long axis of the implant. A screw is then inserted securing the position of the nail in the bone fragment. The process is repeated for the remaining fixation holes located on both sides of a fracture enabling a weight-bearing reduction of the fracture for healing.

Fixation holes on the nail nearest the point of entry into the first bone segment are generally targeted using an outrigger style insertion handle connected to features in the proximal end of the implanted device. The handle comprises apertures colinear with each of the fixation holes the in the proximal end of the nail, and a drill may be advanced through the guide apertures and targeted hole with good reliability. However, targeting the fixation holes in the nail located in the second bone segment can be challenging due to a deflection of the distal end of the nail relative to the proximal end that may occur when placed in the bone. These deflections may be caused by anatomical inconsistencies commonly found in a population and vary due to conditions of rod length or elastic modulus, bone curvature, or other factors. It is generally accepted that targeting the distal fixation holes with an outrigger style drill guide is not reliable, and various solutions have been proposed.

One method of distal targeting relies on an iterative approach using an intra-operative x-ray machine, commonly known as a C-arm, combined with a radio-opaque reference pin. Several images are taken while the surgeon manually aligns the pin to the distal fixation hole in the intramedullary rod, and when the surgeon is confident that the drill axis has been identified, an attempt to drill through the cortex along the centerline of the targeted hole is made. If multiple unsuccessful attempts are made, the cortical bone intended to provide structural support may be rendered insufficient and necessitate exchanging the rod for an alternate implant having fixation screws in other locations. A second problem encountered using this method is the elevated level of radiation exposure experienced by the surgical staff and the patient. Often, lead vests are worn to minimize the radiation exposure, with the extra weight of the vest contributing to fatigue.

Other methods employ the use of magnetic field sensors interacting with a magnetic field enabled to calculate the position of the flux sensor with respect to the field. One example, described in U.S. Pat. No. 8,623,023 B2, couples a drill guide to a magnetic field generator moveable outside the bone which perturbs an array of small coils placed inside the lumen of the nail located in a known position and orientation (pose) relative to the targeted hole. The position of the drill guide-field generator assembly with respect to the sensor can be calculated by the interpretation of the signals generated by the sensor in response to the unique pose in the magnetic field, which is then related to the pose of the guide to the targeted hole by further calculation. A second example described in U.S. Pat. No. 7,060,075 B2 employs similar phenomena by placing wired or wireless magnetic field sensors disposed temporarily in the lumen of the nail or integrated with the body of the nail. Various examples of position sensing by placing a magnetic source inside the lumen of the nail in a known location to a targeted hole while a moveable drill guide coupled to a magnetic flux sensor have also been well described. U.S. Pat. Nos. 5,127,913 and 7,785,330 B3 illustrate examples of permanent magnets coupled in rigid communication with a target while a moveable sensor-drill guide assembly operates to relate the position of the sensor with respect to the magnetic source to the position of the drill guide with respect to the targeted feature. In another example, U.S. Pat. No. 5,584,838 describes an apparatus which places a magnetic field generating coil inside the nail in conjunction with the targeted feature while a sensor array couped to a moveable drill guide.

Though electromagnetic position sensing removes the radiation exposure to the patient and surgical staff, the method presents its own limitations. Metallic objects such as instrumentation or the surgical table located within the magnetic field volume can significantly influence the purity of the data collected by the sensor and contribute to inaccurate positional calculation. Field generation equipment can be large and cumbersome when attached directly to the drill guide, and the latency between movement of field and the updated position on the display can be high. In contrast, magnetic field sources placed within or bonded to the implant are limited in size causing the flux volume to be compact, limiting the sensitivity of the system to minor changes in position of the guide which affects the accuracy of the tool-target alignment.

Other systems and methods have been developed to track the position of a non-visible feature by detecting the deflection of the distal end of the nail, while tracking the proximal end using a secondary navigation system. U.S. Pat. No. 8,382,759 describes a fiducial marker coupled to the proximal end of an intramedullary nail and tracked by an optical navigation system in a coordinate system. A deformation detection device comprising a shape-sensing fiber optic cable is placed in the lumen to provide a measurement of the deflection of the target feature from a first, resting position in the reference frame to a second, deflected position with respect to the tracked fiducial. The deflected position can then be determined in the coordinate system and targeted by a surgical tool tracked in the same reference frame by the navigation system. U.S. Pat. App. Publ. No. 2013/0281884 A1 (filed 23 Apr. 2013) presents a similar method where the proximal end of the intramedullary nail is tracked using a surgical navigation system, however, the deformation detection device placed in the lumen employs electrically powered linear strain sensors. The combination of tracking systems presents the problem of compounding errors which impacts accuracy, as well as the increased cost of providing two measurement systems to track and align a guide to a feature.

Therefore, a clear need exists for a system which improves the process for the targeting of non-visible features during surgical procedures.

### BRIEF SUMMARY OF THE INVENTION

In one embodiment of the invention a system for targeting a feature on a non-rigid surgical device comprises a shape sensing element having a first element end and a second element end and a plurality of nodes dispersed therein, the first element end coupled to an interrogator, wherein the shape sensing element is operable to receive an interrogation signal from the interrogator and return a modified signal to the interrogator related to the pose of each node in a coordinate system, a first node is coupled in rigid communication with a guiding system and at least a second node is coupled in rigid communication with at least one target feature on the non-rigid surgical device, the non-rigid surgical device has a first device end and a second device end, wherein the at least one target feature has a first feature end and a second feature end and a feature axis therebetween, and an interrogator operable to provide the interrogation signal to the shape sensing element and receive the modified signal from the shape sensing element, generate interrogation information related to the position and orientation of the nodes in the coordinate system, and provide the interrogation information to a data processing system, and a data processing system operable to: a) receive the interrogation information from the interrogator, interpret the interrogation information to determine the position and orientation of the nodes in the coordinate system, provide to the user adjustment information related to the change in pose of the feature axis with respect to the first device end, wherein the change in pose of the first node with respect to the second node is related to the change in pose of the feature axis with respect to the first device end in the coordinate system, wherein the guide system is removably coupled to the first device end, and comprises a guide aperture having a first guide end and a second guide and a guide axis therebetween, the guide aperture being moveable with respect to the first device end and operable to pass a surgical tool along the guide axis, the adjustment information is related to a translation of the guide aperture with respect to the first device end such that the guide axis is aligned with the feature axis. The surgical device may be an intramedullary nail, a fixation plate, a portion of a joint reconstruction implant, a second surgical tool, or any combination thereof.

Also in one embodiment the shape sensing element comprises at least one optical fiber, wherein the at least one optical fiber comprises at least one core, wherein the at least one core is operable to conduct the interrogation signal and the modified signal therethrough, wherein the interrogation signal comprises at least one wavelength of light. The shape sensing element may comprise at least one core provided in a helical shape and may include a plurality of Fiber Bragg Gratings dispersed in the at least one core and may be removably coupled to the interrogator.

Also in one embodiment the targeting system further comprises a calibration file containing information related to the position of the target datum in the target reference frame, wherein the calibration file is provided as digital information stored on a portable memory device, or as digital information accessible by the data processing system via a network data connection, or a combination thereof.

Also in one embodiment the display is incorporated into a device wearable by the user.

Also in one embodiment the interrogation information or information related to the comparison may be provided to the data processing system via a wireless data transmission system.

In another aspect of the invention a medical apparatus comprises a surgical device having a first device end and a second device end and having at least one target feature, wherein the at least one target feature has a first feature end and a second feature end and a target axis therebetween, a shape sensing element having a first element end and a second element end and a plurality of nodes dispersed therein, the first element end connectable to an interrogator, wherein the shape sensing element is operable to receive an interrogation signal from the interrogator and return a modified signal to the interrogator related to the position and orientation of each node in a coordinate system, wherein a first node is coupled in communication with the first device end and least a second node is coupled in communication with the at least one target feature, the first device end is connectable to a guide system, the guide system comprises a guide aperture, the guide aperture having a first guide end and a second guide end and a guide axis therebetween, the guide aperture is moveable with respect to the first device end, and information provided to the user related to the change in position and orientation of the second node with respect to the first node is related to a translation of the guide aperture with respect to the first device end such that the guide axis is aligned with the target axis. The medical apparatus may further comprise a calibration file containing information related to the position of the target datum with respect to the first device end, wherein the calibration file is provided as digital information stored on a portable memory device, or as digital information accessible by the data processing system via a network data connection, or a combination thereof. The medical apparatus may be constructed from a composite material

Also disclosed herein is a method of aligning a tool to a target feature on a surgical device, comprising the steps of coupling a first node of a shape sensing element in communication with a first device end of the surgical device, the shape sensing element having a first element end and a second element end and a plurality of nodes therebetween, the first element end coupled to an interrogator, the interrogator operable to provide an interrogation signal to the shape sensing element and receive a modified signal from the shape sensing element related to the position and orientation of each node in a coordinate system and transmit the interrogation information to a data processing system, the data processing system operable to interpret the interrogation information and determine the pose of each node in a coordinate system, coupling a second node of the shape sensing element in communication with at least one target feature on a surgical device, the at least one target feature having a first feature end and a second feature end and a target axis therebetween, coupling a guide system to the first device end, the guide system comprising a guide aperture, wherein the guide aperture is moveable with respect to the first device end and comprises a first guide end and a second guide end and a guide axis therebetween, providing an interrogation signal to the shape sensing element, receiving the modified interrogation signal from the shape sensing element, interpreting the modified interrogation information to determine the pose of each node in the coordinate system, and providing adjustment information to the user related to the change in pose of the second node with respect to the first node, wherein a change in pose of the second node with respect to the first node is related to a change in pose of the target axis with respect to the first device end, the adjustment information is related to the change in pose of the target axis with respect to the first device end, the adjustment information enables the user to move the guide aperture with respect to the first device end such that the guide axis is aligned with the target axis.

### BRIEF DESCRIPTION OF THE DRAWINGS

The foregoing and other objects, features, and advantages will be apparent from the following more elaborate description of the embodiments, as illustrated in the accompanying drawings in which like reference characters refer to the same parts throughout the different views. The drawings are not necessarily to scale, emphasis instead being placed upon illustrating the principles of the embodiments. For a detailed description of example embodiments, reference will now be made to the accompanying drawings in which:

FIG. 1 is a perspective view of an example of a known optical fiber having multiple Fiber Bragg Grating sensors disposed in a single core, in accordance with the disclosure;

FIG. 2 is a perspective view of one example of a known multicore shape-sensing optical fiber, in accordance with the disclosure;

FIGS. 3A-3C each are front and perspective views, respectively, of examples of known configurations of the components of a shape-sensing element, in accordance with the disclosure;

FIG. 4 illustrates an example of a known fiber optic shape sensing system, in accordance with the disclosure;

FIG. 5 is a perspective view of a targeting system for aligning a tool to a feature on a surgical device comprising a shape sensing element, according to an embodiment of the present invention;

FIGS. 6A-6C are perspective, front, top, and section views, respectively, of the targeting sensor of FIG. 5, according to an embodiment of the present invention;

FIG. 7A illustrates front and section views of a portion of the targeting sensor of FIG. 6C, according to an embodiment of the present invention;

FIG. 7B illustrates front and section views of a portion of the probe of FIG. 5, according to an embodiment of the present invention;

FIG. 8A is an exploded view of the intramedullary nail and hole targeting assembly of FIG. 5, according to an embodiment of the present invention;

FIG. 8B illustrates perspective and front views of the drill guide assembly of FIG. 8A, according to an embodiment of the present invention;

FIG. 8C illustrates perspective view of the hole targeting assembly of FIG. 5 coupled to an intramedullary fixation device, according to an embodiment of the present invention;

FIG. 9 illustrates a top, side, and section view of the hole targeting assembly and the targeting sensor coupled to an intramedullary nail and configured for calibration, according to an embodiment of the present invention;

FIG. 10 shows a perspective view of the targeting system of FIG. 5 configured for calibration, according to an embodiment of the present invention;

FIG. 11 is a perspective view illustrating the configuration of the targeting system of FIG. 5 during the implantation step of an intramedullary nail into a fractured tibia, according to an embodiment of the present invention;

FIGS. 12A-12B are perspective and detail view of the targeting system of FIG. 5 configured for targeting a distal fixation hole of an implanted intramedullary nail, according to an embodiment of the present invention;

FIG. 12C is a side view of the distal portion of a tibia coupled to the targeting system of FIG. 5 configured to target a distal fixation hole in an implanted intramedullary nail, according to an embodiment of the present invention;

FIG. 13A is a schematic diagram of the targeting system of FIG. 5, according to an embodiment of the present invention;

FIG. 13B is a schematic diagram of the control unit of FIG. 13A, according to an embodiment of the present invention;

FIG. 14 is a workflow diagram of a calibration process, according to an embodiment of the present invention;

FIG. 15 is a perspective view of a targeting system for aligning a tool to a feature on a surgical device comprising a shape sensing element, according to a second embodiment of the present invention;

FIGS. 16A-16B are perspective views of an intramedullary nail integrated with a targeting sensor, according to an embodiment of the present invention;

FIG. 16C illustrates section views of the assembly of FIG. 16A, in accordance with the disclosure;

FIG. 16D illustrates a section view of an alternate means of bonding the shape sensing element of FIG. 16A to an intramedullary nail, according to an embodiment of the present invention;

FIG. 17 illustrates a section view of an alternate means of bonding the shape sensing element of FIG. 16A to a composite intramedullary nail, according to an embodiment of the present invention;

FIG. 18 is a perspective view of the intramedullary nail and targeting sensor of FIGS. 16A-16D implanted into a fractured tibia, according to an embodiment of the present invention;

FIG. 19 is a perspective of the targeting system of FIG. 16 configured for targeting a distal fixation hole of an implanted intramedullary nail, according to an embodiment of the present invention;

FIG. 20 is a workflow diagram of a targeting process, according to an embodiment of the present invention.

### DETAILED DESCRIPTION

While the invention is amenable to various modifications, permutations, and alternative forms, specific embodiments have been shown by way of example in the drawings and are described in detail below. The intention, however, is not to limit the invention to the embodiments described. The invention is intended to cover all modifications, equivalents, and alternatives falling within the scope of the invention as defined by the appended claims.

The inventor provides a system for aligning a surgical tool to a feature on an orthopedic device by employing a shape-sensing cable in a novel manner. One commercially available technology capable of providing a dynamic reconstruction of the shape of a cable in a coordinate system comprises a single optical fiber or a collection of optical fibers coupled to an interrogator and data processing unit. Sets of optical strain sensors disposed within multiple cores of a single fiber, or the cores of a bundle of single-core fibers, which transmit and selectively reflect light provided by the interrogator, are interpreted to generate 3-dimensional position and orientation data of nodes along its length in a coordinate system. A brief discussion of the general concepts of fiber optic shape sensing (FOSS) is now provided to help illustrate various implementations of the present invention.

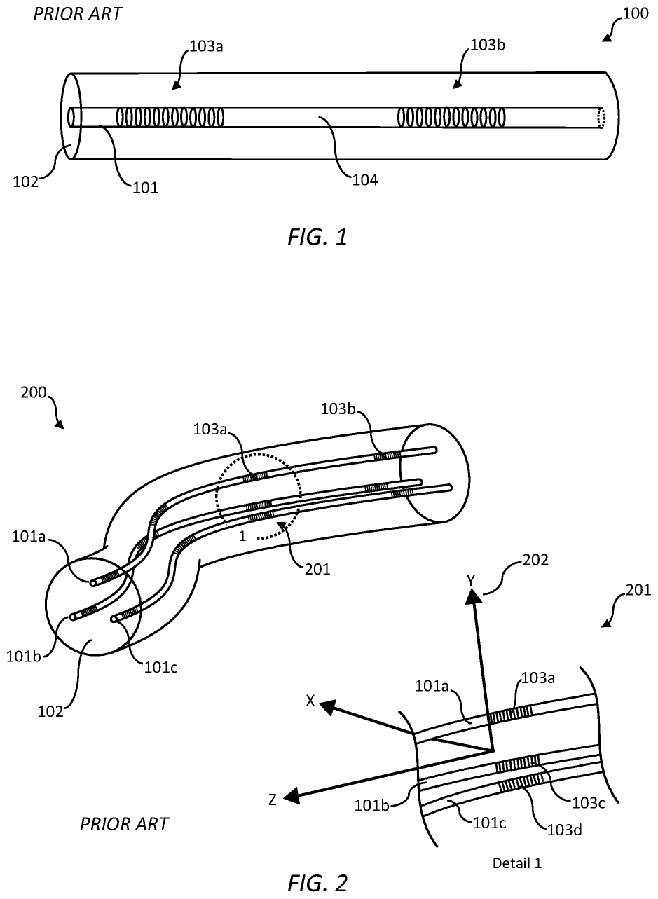

FIG. 1 illustrates the basic construction of a single-core optical fiber 100. A light conducting core 101 is shown formed in the interior of a cladding 102. As a light signal propagates along core 101, particular wavelengths are scattered by random imperfections or reflected at a defined sensor point by formed features, known as Fiber Bragg Gratings (FBGs), while other wavelengths pass unaffected. Changes in pressure, strain, or temperature can change the refractive index of the sensor point thereby altering the wavelength reflected back to the source, generally known as the Bragg wavelength. Core 101 is shown having a first strain sensor 103a separated from a second strain sensor 103b by a tether 104 which has no signal changing properties. A protective coating 105 (not shown for clarity) is generally bonded to the outer surface of cladding 102.

An example of a multicore optical fiber 200 having a first core 101a, a second core 101b, and a third core 101c formed in a common cladding 102 and dispersed evenly from the central axis of the fiber as shown in FIG. 2. As a portion of the fiber experiences a bending force, a measurable strain field develops such that some areas of a cross section may be tension while others are in compression, depending on the direction of the bend. A set of strain sensors 103a, 103c, and 103d form a node 201 having a reference frame 202 which has a calculable position and orientation with respect to a neighboring node when the strain data from each sensor is interpreted. As the number of nodes increases in a length of shape-sensing cable, the tether portion reduces in length and the positional resolution improves. Fully distributed FBGs provide a scheme for continuous measurement along the cable and are well described.

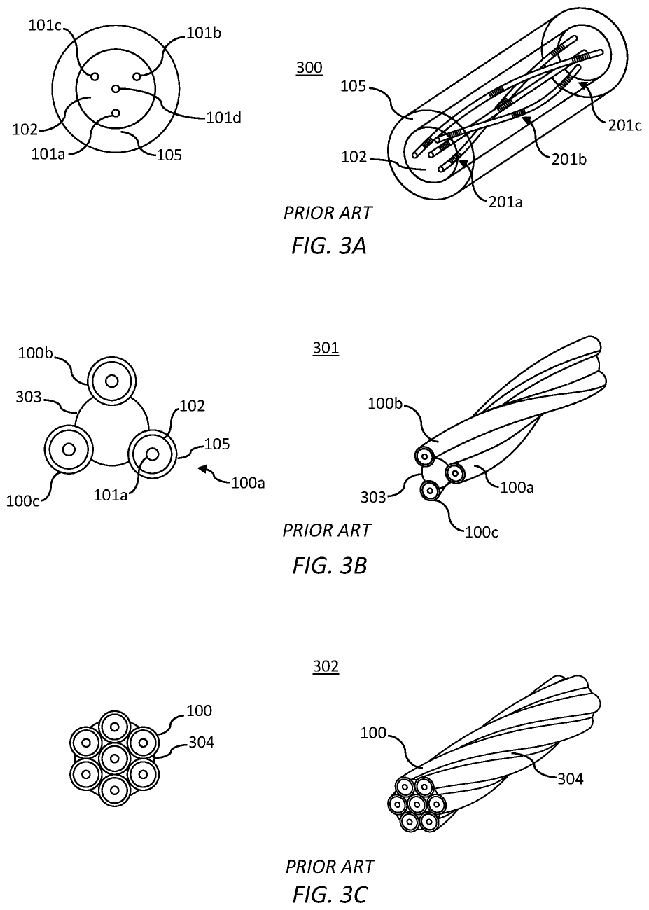

Various configurations of the cores of the shape sensing element have been described to improve positional resolution, accuracy, and the sensitivity to twist around the central axis of the cable with relevant examples shown in FIGS. 3A-3C. FIG. 3A shows a multi-core optical fiber 300 having several cores dispersed in a common cladding. Cores 101a-101c are dispersed in a helical pattern around a fourth core 101d which is placed colinear with the central axis of the fiber with coating 105 bonded to the outer surface of cladding 102. Sets of FBG strain sensors arranged within the cores form nodes 201a-201c at intervals along the length of the cable. FIG. 3B shows a shape sensing element 301 comprising three single-core optical fibers 100a-100c bonded to the outer surface of a central element 303 and arranged in a helical pattern about the central axis of the cable. In another configuration, several single-core optical fibers 100 are twisted together in a helical pattern an bonded together using an adhesive 304 to form a multi-fiber bundle 302 shown in FIG. 3C. One advantage of the multifiber bundle approach is the ability to arrange the FBG sensors in various configurations where an improvement in accuracy and sensitivity for a given number of sensors may be realized.

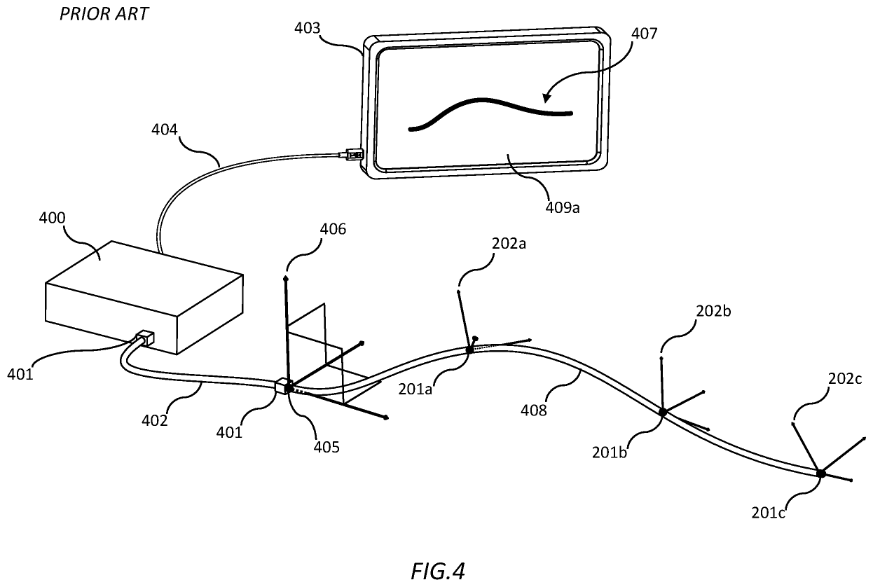

FIG. 4 shows the major components of a commercially available fiber optic shape-sensing system which can be deployed to quantify the dynamic changes in shape of an object couped to the shape-sensing element or track the position of objects connected to various nodes in real time within a coordinate frame. An interrogator 400 is connected to a patch cable 402 through a multicore coupler 401 at the first end, while a second multicore coupler 401 at the second end of patch cable 402 connects to a shape sensing element 408. Patch cable 402 comprises an equivalent number of cores as shape sensing element 408, though it does not contain any strain sensors and operates to transmit light signals over a length of cable where shape-sensing is not required. Thus, interrogator 400 may be placed at a distance, such as outside a sterile field in an operating theater, from the first shape-sensing node and allowing the limited number of nodes to be concentrated in the length of cable where shape-sensing is desired to maximize accuracy and sensitivity.

Interrogator 400 is a data acquisition component that provides an outbound interrogation signal in the form of a light signal to each core, in either a multicore fiber or multifiber bundle, and receives an inbound interrogation signal reflected light back from the FBGs or other reflective elements embedded in the core for interpretation. Various techniques of interrogation are available to generate the data necessary to calculate a shape reconstruction 407, with non-limiting examples being Wavelength Division Multiplexing (WDM), Optical Frequency Domain Reflectometry (OFDR), and Optical Time Domain Reflectometry (OTDR). The first end of shape sensing element 408, connected to patch cable 402, has a base reference frame 406 associated with a base node 405. Base reference frame 406 may be a Cartesian coordinate system in which the position and orientation of all other nodes of the shape sensing element may be defined. For illustration purposes, a first calculated node 201a has a position (X₁, Y₁, Z₁) and an orientation defined by a first calculated reference frame 202a. A second reference frame 202b and third reference frame 202c downstream from node 201a have a unique coordinate positions (X₂, Y₂, Z₂ and X₃, Y₃, Z₃, respectively) in base reference frame 406 with a reference frame 202b and a reference frame 202c, respectively, defining their orientations. A data connection cable 404 may provide a power source and a data connection to transfer the information collected by interrogator 400 to the data processing equipment in a control unit 403 where shape reconstruction 407 is calculated and rendered as an image provided to the user on a display 409a.

The present invention is now described in enabling detail in the following examples, which may represent more than one embodiment. Although one or more of these embodiments may be preferred, the examples disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment. It should be noted that for the purposes of this disclosure, pose may be defined as the position or orientation or combination thereof of an object, feature, or datum in a coordinate system or with respect to other objects, features, or datums.

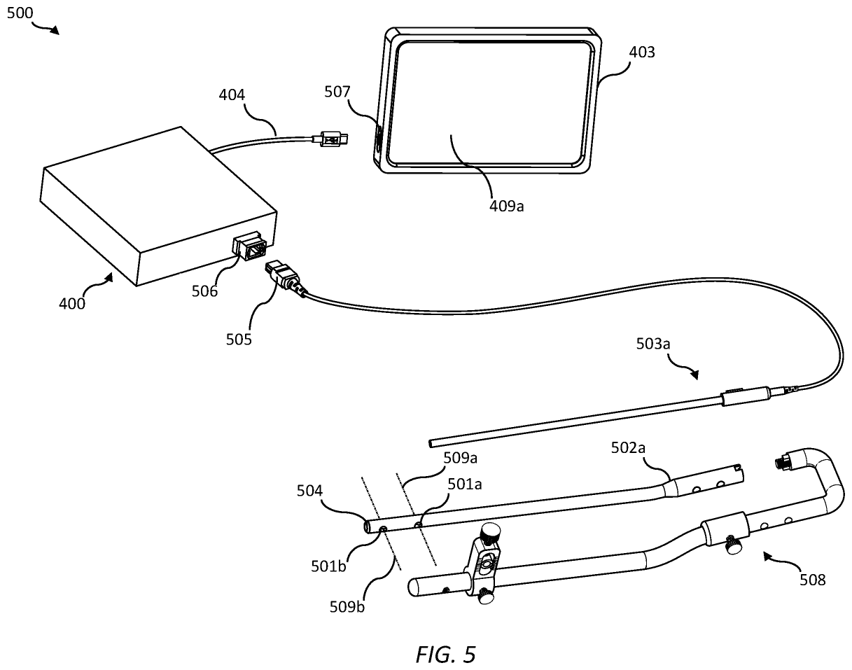

Referring now to FIG. 5, one embodiment of a targeting system 500 is shown in perspective view, and is designed to provide the user a means of aligning a surgical tool to a first distal fixation hole 501a having a target axis 509a, and a second distal fixation hole 501b having a target axis 509b, in an intramedullary nail 502a having a central lumen 504. In this example, targeting system 500 comprises a targeting sensor 503a, control unit 403 connectable to interrogator 400 by data connection cable 404, and a guide system 508. In this embodiment, display 409a is a touchscreen monitor integrated with control unit 403 operating as a graphical user interface (GUI) to the system's software, and for visual presentation of information to the user during the surgical procedure, having a screen of sufficient size enabling the user to view the data when placed outside of the sterile field. A port 507 provided with control unit 403 may supply power to interrogator 400 as well as a data link for the transfer of interrogated information or commands between the two components via data connection cable 404. Several types of powered, data connection devices are currently available which may include, but are not limited to universal serial bus (USB), serial-ATA (SATA), or peripheral component interface (PCI). Non-powered, signal-only connection may also be used in certain configurations where interrogator 400 and control unit 403 are provided with separate power sources. Targeting sensor 503a is operationally connected to interrogator 400 using commercially available MPO/MTO multicore fiber optic connectors. A male multicore connector 505 is affixed at a first end of targeting sensor 503a connectable to a female multicore connector 506 supplied on interrogator 400 such that light signals provided by the interrogator are made available to targeting sensor 503a for interrogation.

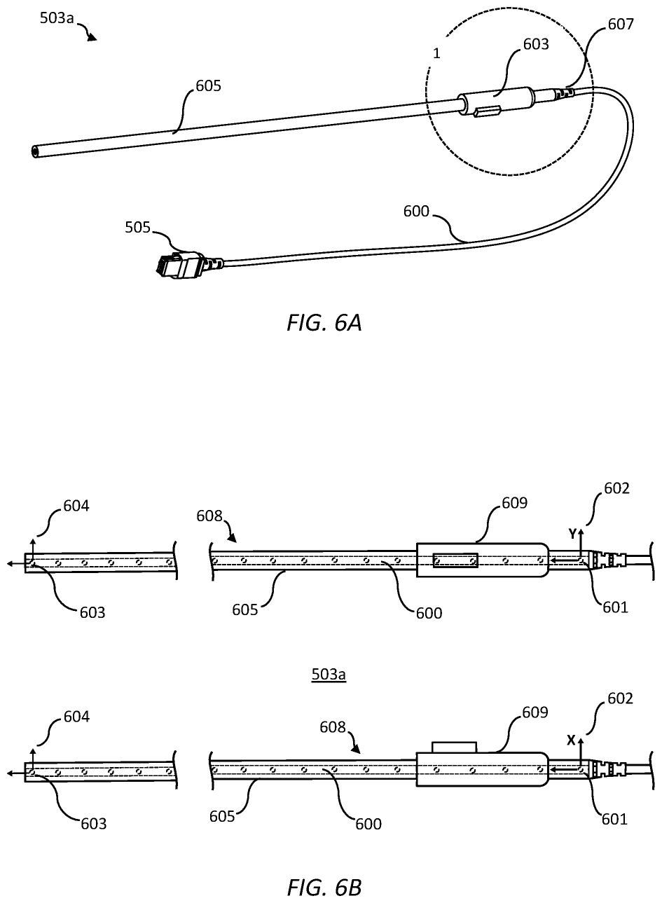

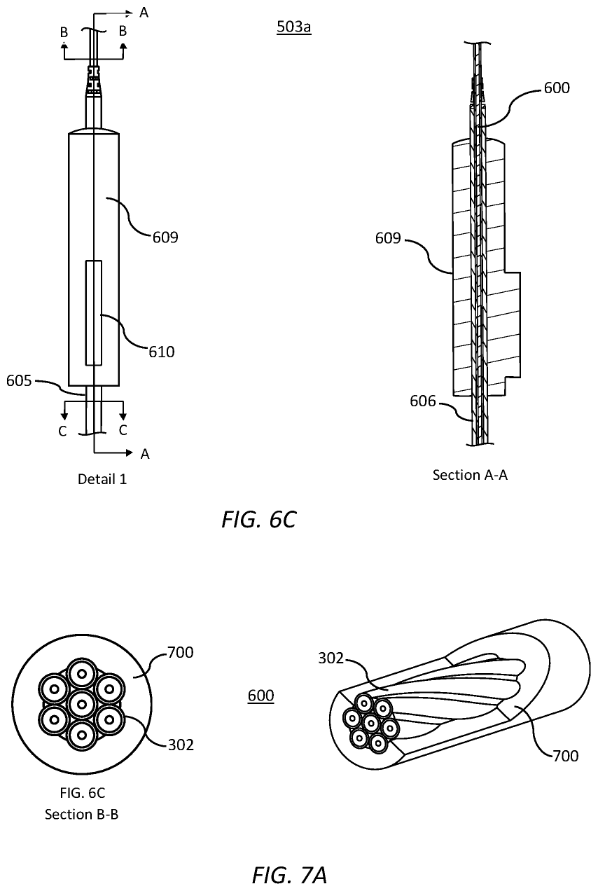

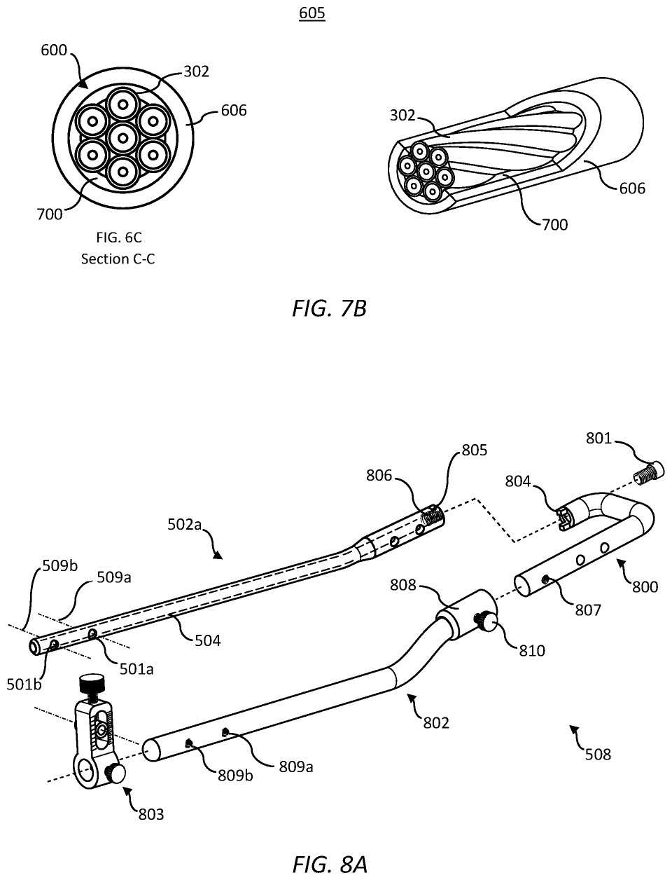

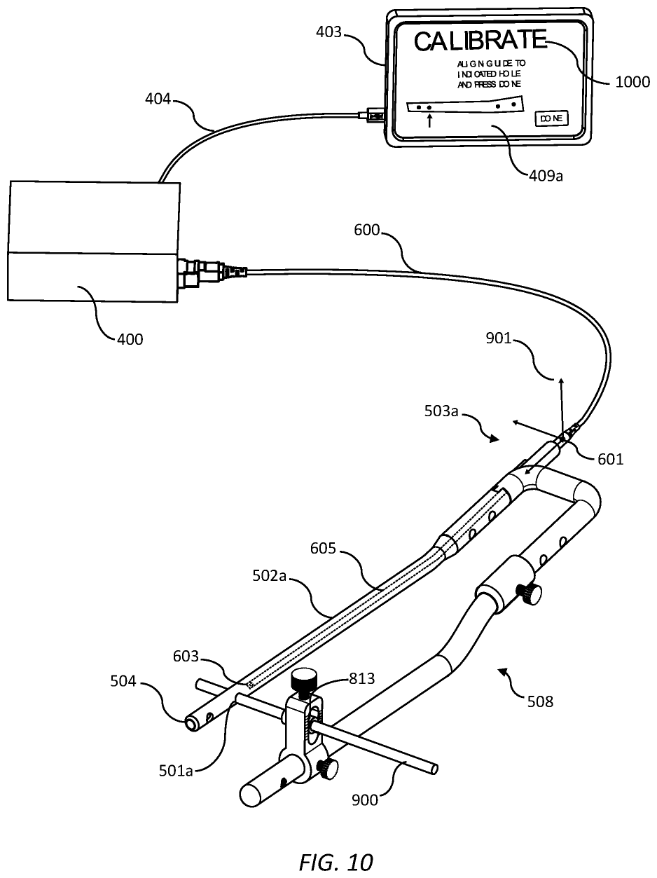

FIGS. 6A-6C are perspective, top side, and section views, respectively, of targeting sensor 503a, according to an embodiment of the present invention. Targeting sensor 503a is designed to be placed in the central lumen of an intramedullary nail and removably and rigidly couple a base node 601 in a first portion of a shape sensing cable 600, having a base reference frame 602, to the proximal end of intramedullary nail 502a, and removably and couple a target node 603 in a second portion of shape sensing cable 600, having a target reference frame 604 to the distal portion of intramedullary nail 502a in a fixed and repeatable location with respect to distal fixation holes 501a and 501b such that target node 603 will experience the same amount of deflection with respect to the proximal end of intramedullary nail 502a as distal fixation holes 501a and 501b due to nail bending. In this embodiment, shape sensing cable 600 employs a length of multi-fiber bundle 302 (illustrated in FIGS. 7A-7B) connectable to interrogator 400 via a male multicore connector 507 affixed at the first end, with a portion extending through the center of a probe 605, where interrogator 400 is placed outside the sterile field. Shape sensing cable 600 is provided operable to receive light signals from interrogator 400 and reflect light information from the plurality of FBG sensor points in each core of multi-fiber bundle 302, forming a set of nodes 608 between base node 601 and target node 603, to enable the localization of target node 603 in base reference frame 602. Probe 605 is designed to include the portion of shape sensing cable 600 containing the set of nodes 608 and is introducible into lumen 504 to enable the placement of target node 603 in a fixed location substantially close to but without obscuring the proximal-most distal fixation hole of the intramedullary nail. A sleeve 606 encapsulates shape sensing cable 600 proximal to base node 601 and extends towards target node 603. Sleeve 606 is an elastically deformable tube designed to add rigidity and protect shape sensing cable 600 and may be constructed using a variety of biocompatible materials with examples including but not limited to alloys of stainless steel, alloys titanium, nitinol or zirconium. Shape sensing cable 600 may be secured in the interior of sleeve 606 using an adhesive, a crimp, or an interference fit. Sleeve 606 may also be formed by over-moulding shape sensing cable 600 with biocompatible thermoplastics such as polyether-ether-ketone (PEEK), thermoset plastics such as polyurethane, or composites of high strength fiber and a thermoset or thermoplastic matrix. A stress relief 607 may be affixed to the proximal end of probe 605 to provide mechanical support and promote a smooth radius of curvature of the extending portion of shape sensing cable 600. A handle 609 is formed at the proximal end of probe 605 having a key 610 to fit with mating features in guide system 508 (shown in FIG. 10) to rigidly couple base node 601 in a reproducible location with respect to the proximal end of intramedullary nail 502a.

FIGS. 7A-7B are section views of shape sensing cable 600 and probe 605, respectively, where their components may be further appreciated. As shown in FIG. 7A, shape sensing cable 600 may be constructed with multi-fiber bundle 302 as the shape sensing core of the cable, and a coating 700 bonded to add strength and stiffness to the cable for protection and handling properties. Examples of suitable biocompatible materials in coating 700 may include, but are not limited to medical grades of polyvinylchloride, polyethylene, PEEK, polycarbonate, polyetherimide, polysulfone, polypropylene, or polyurethane. FIG. 7B shows a cross-section of a portion of shape sensing cable 600 that is encapsulated in sleeve 606 to form the structure of probe 605.

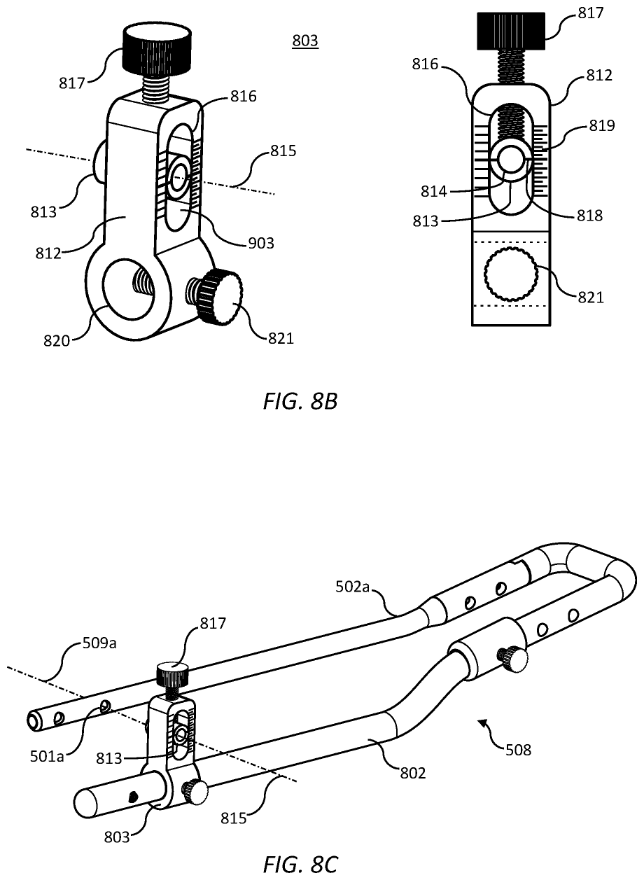

FIGS. 8A-8C illustrate the components of a guide system 508 designed to couple with the proximal end of intramedullary nail 502a, according to an embodiment of the present invention. In this example, guide system 508 is designed to enable a drill guide assembly 803 having an adjustable drill guide 813 to be removably coupled in rigid communication to the proximal end of intramedullary nail 502a. An insertion handle 800 is secured to intramedullary nail 502a using a cannulated bolt 801, connectable to a thread 806 formed in the proximal end intramedullary nail 502a and operable to provide access to lumen 504. Insertion handle 800 is a “U”-shaped tubular body having a key 804 formed on one leg designed to engage with a slot 805 formed in the proximal end of intramedullary nail 502a to provide rotational stability with no degrees of freedom of motion with respect to the proximal end of intramedullary nail 502a. On the opposite leg, a threaded aperture 807 is formed to enable an extension 802 to be rigidly connected thereto using a set screw 810. Extension 802 is an elongate body having a coupler 808 at the proximal end and a first threaded aperture 809a and a second threaded aperture 809b formed in the distal end. Extension 802 is designed to removably couple with insertion handle 800 with the engagement of set screw 810 into aperture 807 providing a secure and rigid connection enabling apertures 809a and 809b to be in rigid communication with the proximal end of intramedullary nail 502a. As shown in FIG. 8B, drill guide assembly 803 is shown in perspective and front view, and is comprised of a body 812 having a mounting aperture 820 formed to provide a close sliding fit with extension 802. A set screw 821 is designed to engage with aperture 809a or aperture 809b and position drill guide 813 parallel with distal fixation hole 501a or distal fixation hole 501b, respectively. Drill guide 813 is adjustable in a slot 816 in a direction parallel to the plane of deflection of intramedullary nail 502a by the rotation of an adjustment screw 817, and is provided with a guide aperture 814 operable to guide a surgical drill along a guide axis 815. An indicator 818 is provided on drill guide 813 and a scale 819 is provided on a viewable surface of body 812 to provide the user with positional information related to the adjustment of drill guide 813 in body 812. As shown in FIG. 8C, guide system 508 is coupled to intramedullary nail 502a and drill guide assembly 803 assembled onto extension 802 and drill guide 813 adjusted such that guide axis 815 is aligned colinear with target axis 509a.

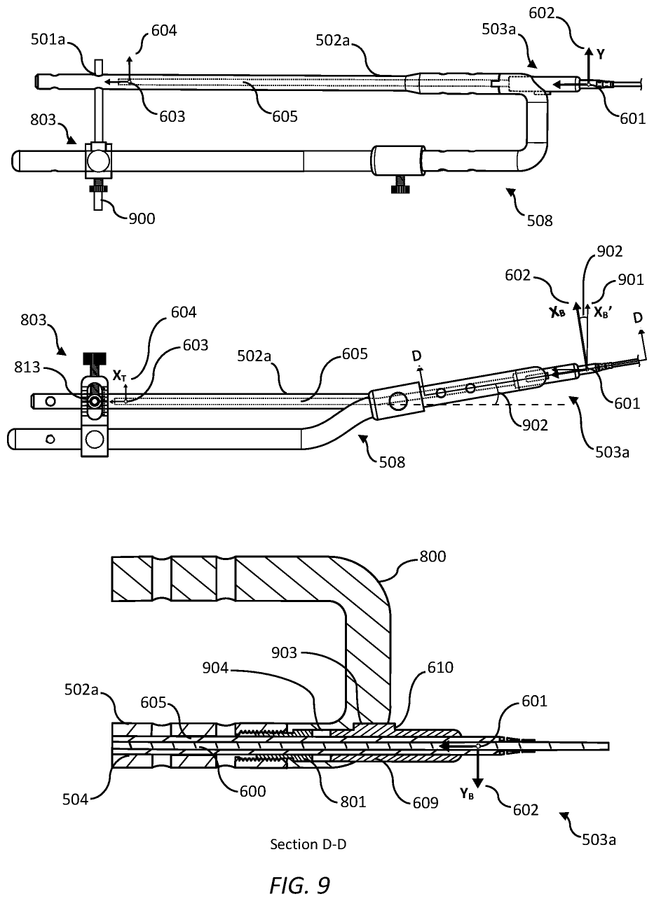

FIG. 9 illustrates a top, side, and section view showing targeting sensor 503a coupled to intramedullary nail 502a, according to an embodiment of the present invention. With guide system 508 assembled onto to the proximal end of intramedullary nail 502a as described in FIGS. 8A-8C, probe 605 is inserted into lumen 504 such that its distal end of arrives immediately proximal to distal fixation hole 501a. Section D-D illustrates the engagement of handle 609 in a positioning aperture 904 formed in insertion handle 800 with key 610 engaged in a slot 903 to locate base node 601 at a fixed position with respect to insertion handle 800 as well as the proximal end of intramedullary nail 502a. In one embodiment, a first position of drill guide 813 in drill guide assembly 803 with respect to base reference frame 602 may be determined by rotating adjustment screw 817 until an alignment pin 900 can be inserted through both drill guide 813 and distal fixation hole 501a while intramedullary nail 502a is in a non-flexed condition. It should be noted that in this configuration, target node 603 is coupled in communication with distal fixation hole 501a, and will move in concert with distal fixation holes 501a and 501b as the nail is flexed. Shape sensing cable 600 is then operationally connected to interrogator 400 and the system initiated to enable the position and orientation of target node 603 to be calculated in base reference frame 602. A skilled artisan will recognize that a variety of intramedullary nails are available having various bends from the proximal end to the distal end to accommodate anatomical features of the bone requiring stabilization. In this example, intramedullary nail 502a is designed for internal fixation of a fractured tibia with a bend at the proximal end at a neck angle 902 with respect to the distal end. When inserted into lumen 504, probe 605 deflects from its straight position at rest to conform to the bend of intramedullary nail 502a also causing a change in orientation of target reference frame 604 where the Z_{T} axis orients parallel to lumen 504, the X_{T} axis is oriented parallel to the adjustment direction of drill guide 813, and the Y_{T} axis is parallel to both guide axis 815 and target axis 509a. A secondary reference frame 901 (X_{B}′, Y_{B}′, Z_{B}′) may be defined by rotating base reference frame 602 about the Y_{B} axis such that X_{B}′ is parallel to X_{T}. As intramedullary nail 502a conforms to the canal of the tibia, bending in the X_{B}-Z_{B} plane may occur. Thus a translation of target node 603 in the X_{T}/X_{B}′ direction may be calculated and provided to the user to represent an adjustment value of drill guide 813 to re-align guide axis 815 to target axis 509a for guided drilling.

FIG. 10 is a perspective view illustrating targeting system 500 configured for calibrating the position of target node 603 to the position of drill guide 813, according to an embodiment of the present invention. As shown, targeting sensor 503a is coupled to intramedullary nail 502a as described heretofore, and is operable to receive an interrogation signal provided by interrogator 400 and return a modified signal to interrogator 400 to generate positional data of each node, which may be referred to as interrogation information. A calibration program 141 program 1000 is loaded into the operating system of control unit 403 with instructions provided to the user on display 409a. The user may select the targeted hole corresponding to the placement of alignment pin 900 in intramedullary nail 502a. With drill guide 813 aligned with distal fixation hole 501a, shape sensing cable 600 is interrogated and the position of target node 603 in secondary reference frame 901 is calculated and recorded.

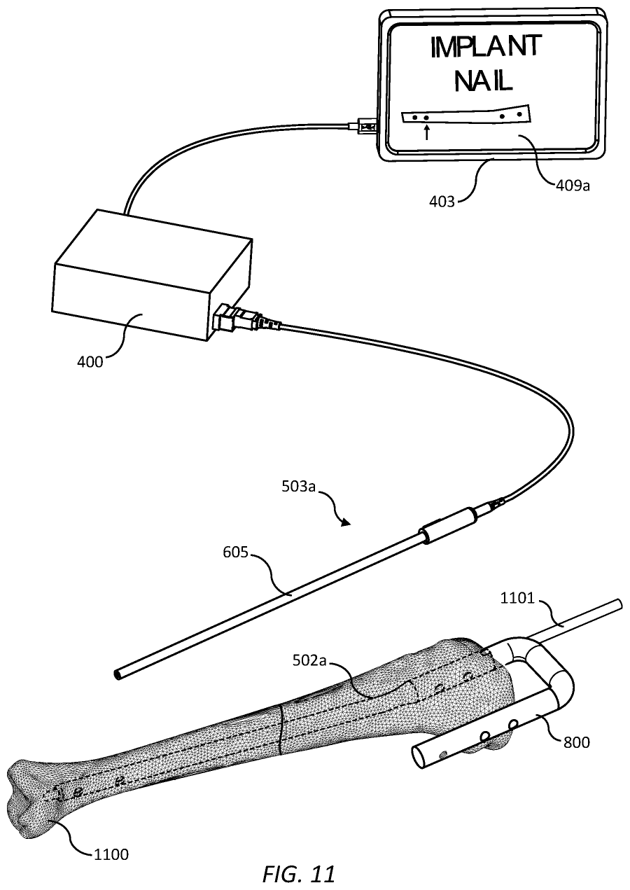

FIG. 11 is a perspective view illustrating the configuration of targeting system 500 during the implantation step of intramedullary nail 502a into a fractured tibia 1100, according to an embodiment of the present invention. In this example, extension 802 is removed from insertion handle 800 enabling the user improved control to manipulate intramedullary nail 502a during the implantation. Targeting sensor 503a is removed from lumen 504 enabling intramedullary nail 502a to be advanced in the intramedullary canal of tibia 1100 from the proximal portion, across the fracture line, and into the distal portion with a guidewire 1101 in lumen 504 serving as a guide. Once the user is satisfied with the position of intramedullary nail 502a in tibia 1100, guidewire 1101 may be removed from lumen 504.

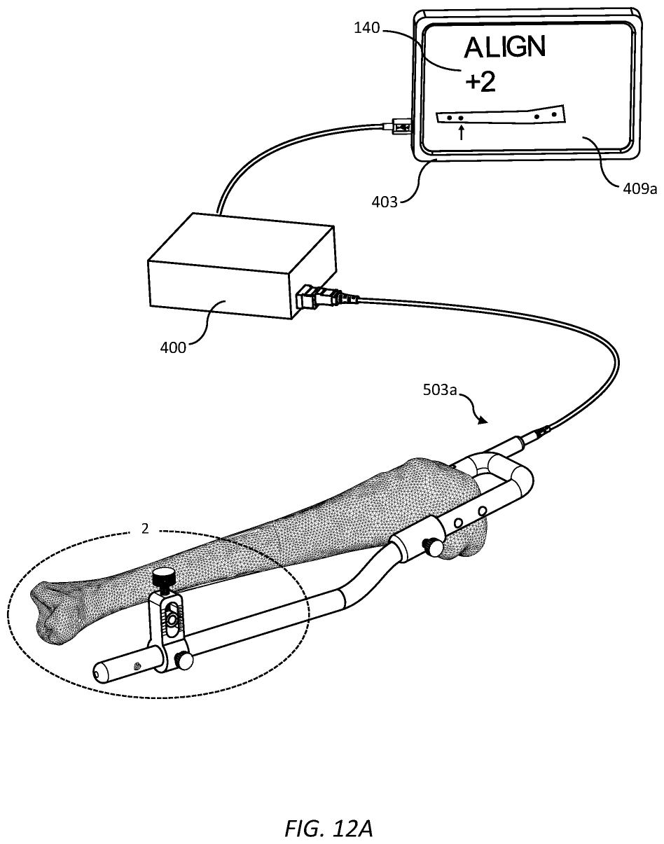

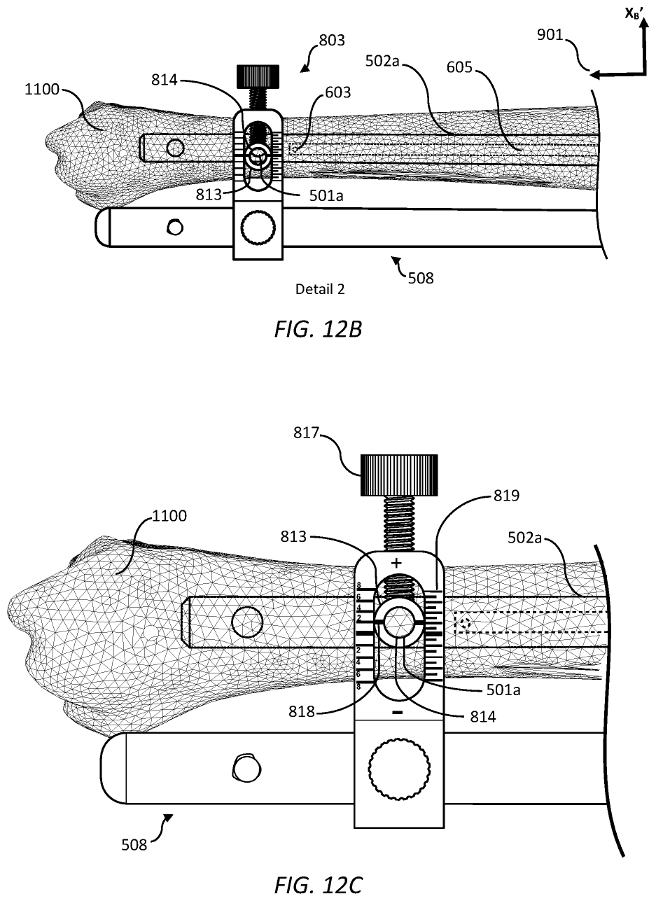

FIGS. 12A-12B are perspective and detail view of targeting system 500 configured for targeting distal fixation hole 501a of implanted intramedullary nail 502a, according to an embodiment of the present invention. In this illustration, distal fixation holes 501a and 501b are obscured from view by the cortex of tibia 1100 as well as the surrounding soft tissue of the lower leg and may have changed shape to conform to the particular anatomy, resulting in a translation of distal fixation hole 501a to a deflected position in secondary reference frame 901. As shown in FIG. 12B, the deflection of the distal end of intramedullary nail 502a with respect to the proximal end results in a misalignment between drill guide 813. Since probe 605 is designed with a close sliding fit in lumen 504, target node 603 is deflected in the same direction by a substantially equal amount. Shape sensing cable 600 is then interrogated and a second position of target node 603 is calculated. The difference in position of target node 603 from the calibrated position to the implanted position in the X_{B}′ direction yields a distance in the X_{B}′ direction that drill guide 813 must be adjusted to align guide aperture 814 to distal fixation hole 501a. The adjustment information may then be provided to the user as a targeting graphic 140 on display 409a. As shown in FIG. 12C, drill guide 813 is adjusted by operating adjustment screw 817 such that indicator 818 aligns with the value on scale 819 as indicated in targeting graphic 140. Guide aperture 814 is now aligned with distal fixation hole 501a for guided drilling.

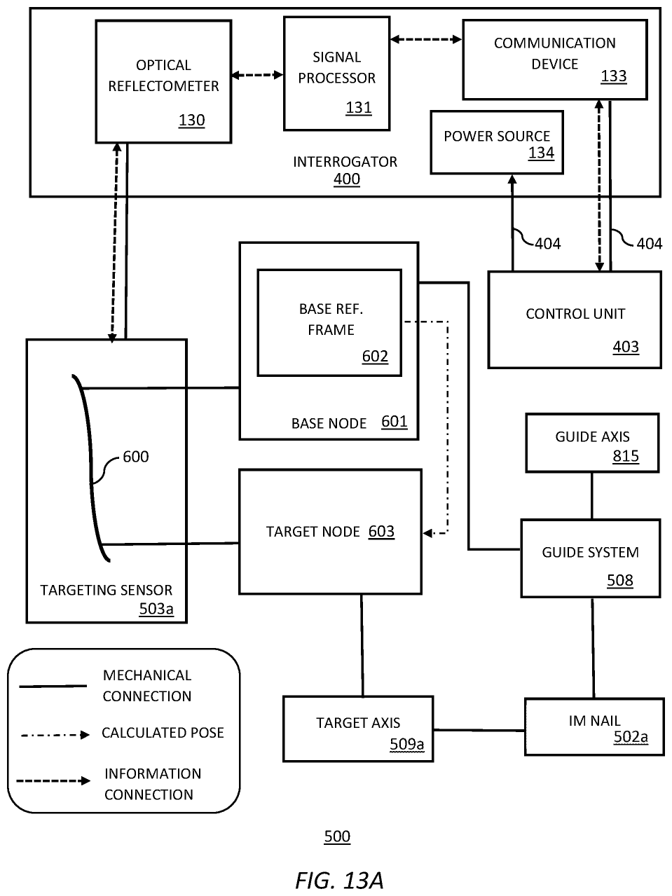

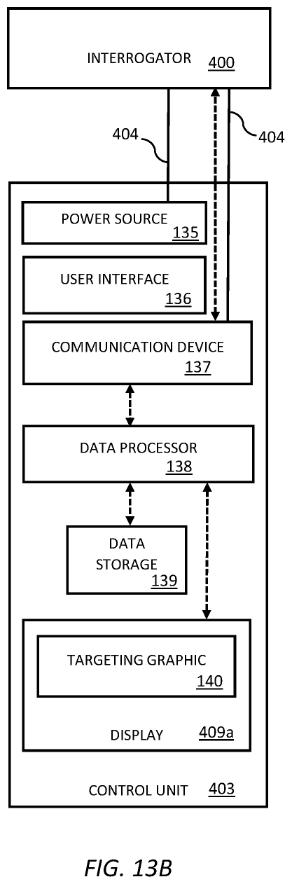

Referring now to FIGS. 13A-13B where diagrams of the major components and connections of targeting system 500, as described heretofore, are illustrated in schematic form, in accordance with the disclosure. In this example, interrogator 400 is provided comprising an optical reflectometer 130 operable to send and receive light signals to the multiple cores of shape sensing cable 600. Using Optical Time Domain Reflectometry (OTDR), Optical Frequency Domain Reflectometry (OFDR), Wavelength Division Multiplexing (WDM), or other suitable interrogation technique capable of providing information relating to the localized strain of a core of an optical fiber, the light signals are processed for the calculation of positional data of each node by a signal processor 131 and transmitted by a communication device 133 to control unit 403 where the computation steps needed for shape reconstruction and node pose of targeting sensor 503a in base reference frame 602 are performed by a data processor 138. Data connection cable 404 provides electrical power to a power source 134 and a data connection link between interrogator 400 and control unit 403. Control unit 403 further comprises a power source 135 and a communication device 137 operable to provide power to and transfer data to and receive data from interrogator 400 via data connection cable 404. A user interface 136 is provided to enable the user to communicate with control unit 403 which may include but are not limited to a wired or wireless keyboard, mouse or other pointing device, a touchscreen display, voice recognizing interface or other interface enabling the user to send commands to control unit 403. A device for data storage 139 accessible by data processor 138 may be provided as random access memory (RAM), read-only memory (ROM), flash memory, erasable program read-only memory (EPROM), or a combination thereof. The comparison of target node 603 in base reference frame 602 from a first, non-flexed position to a second, implanted position can be calculated by data processor 138 and rendered for a visual presentation to the user as a targeting graphic 140 on display 409a. It should be noted that the computation, communication, and interrogation devices and techniques described heretofore are well-known to a skilled artisan with further details of subcomponents or operation omitted for brevity.

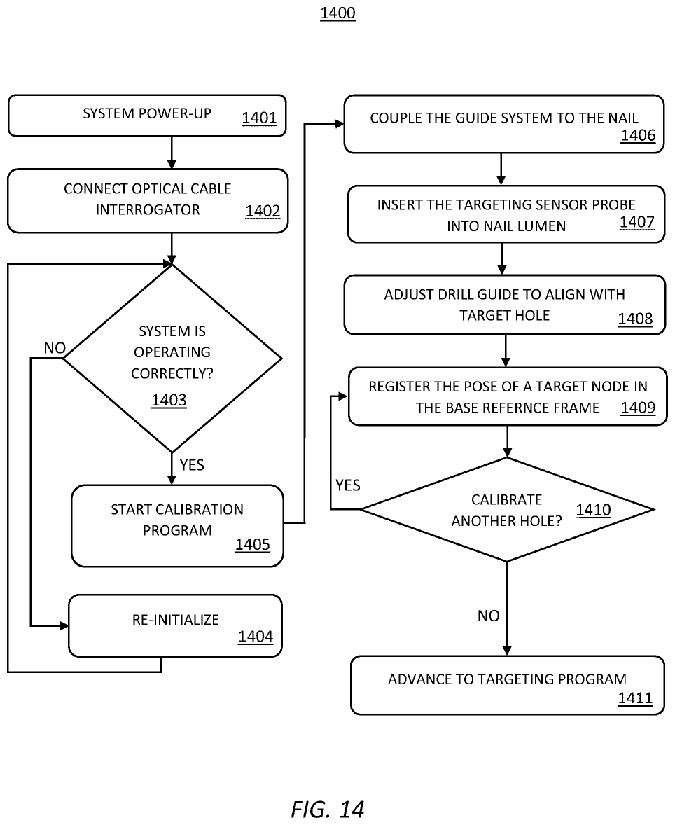

A workflow diagram is presented in schematic form in FIG. 14 to illustrate a method 1400 for calibrating targeting system 500 to a distal fixation hole in an intramedullary nail, according to an embodiment of the present invention. The method begins with step 1401 where the electrical power and communication connections are made, and the system power is turned on. In step 1402, the user connects targeting sensor 503a to interrogator 400. In step 1403, a system check is performed to ensure all power and optical connections are correct and the system is performing the necessary activities for shape-reconstruction of the targeting sensor. If the system is not performing normally, a re-initialization step 1404 is needed where the user would troubleshoot the problem and perform a subsequent system check step 1403. If the system is operating normally, the user may then proceed to step 1405 where a calibration program is loaded into system memory. In step 1406, guide system 508 is affixed to the proximal end of intramedullary nail 502a as described in FIGS. 8A-8C. In step 1407, targeting sensor 503a is coupled to intramedullary nail 502a by inserting probe 605 is inserted into lumen 504 as shown in FIG. 9. This couples target node 603 in rigid communication with distal fixation hole 501a and couples drill guide 813 in communication with base reference frame 602. In step 1408, the user will mechanically align drill guide 813 to the fixation hole selected in the calibration software and indicated on display 409a as described in FIG. 9. In step 1409, shape sensing cable 600 is interrogated to determine a first position of target node 603 in base reference frame 602. The first position is recorded for recall in a later step. In step 1410, the user may either choose to calibrate the system to target another feature, such as distal fixation hole 501b. If this option is chosen, the user will select a different fixation hole in the software and reposition alignment pin 900 and drill guide assembly 803 appropriately, and continue to register another location of target node 603 in base reference frame 602. Once the user has completed the calibration for all desired fixation holes, the process is advanced to step 1411 where the user commands the system to exit the calibration program and load a targeting program into system memory.

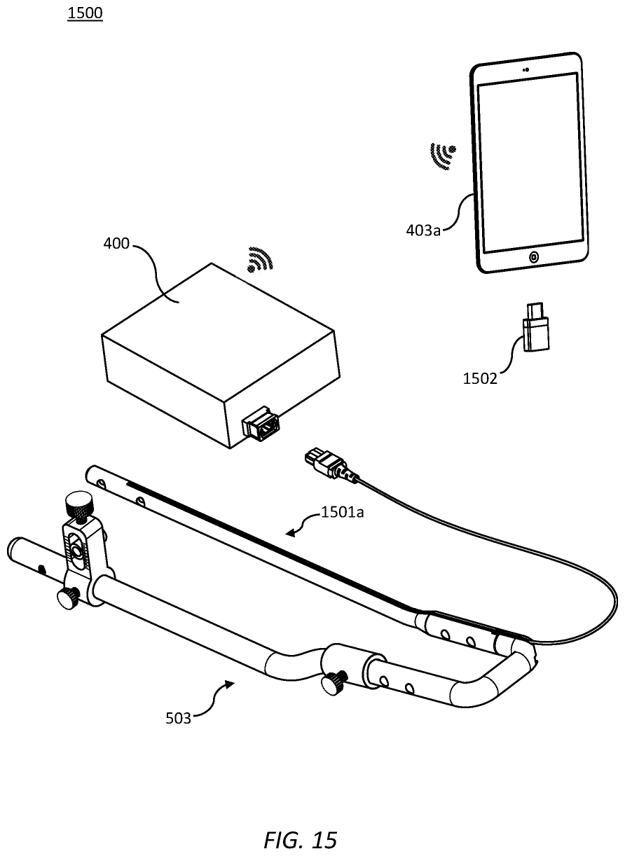

Referring now to FIG. 15, a perspective view of a targeting system 1500 is shown, according to a second embodiment of the present invention, comprising a shape sensing intramedullary nail 1501a connectable to guide system 508 and interrogator 400. In this example, calibration information is provided to the user on a portable memory 1502 which is connectable to a portable control unit 403a control unit 403a. Interrogator 400 may be provided with wireless data transfer equipment to transfer information to and from control unit 403a. Non-limiting examples include but no such as Bluetooth or wireless local area network protocols such as IEEE 802. Portable memory 1502 may be provided as flash memory, RAM, ROM, EPROM, optical storage, or other computer storage media connectable and operable to transfer electronic data to control unit 403a.

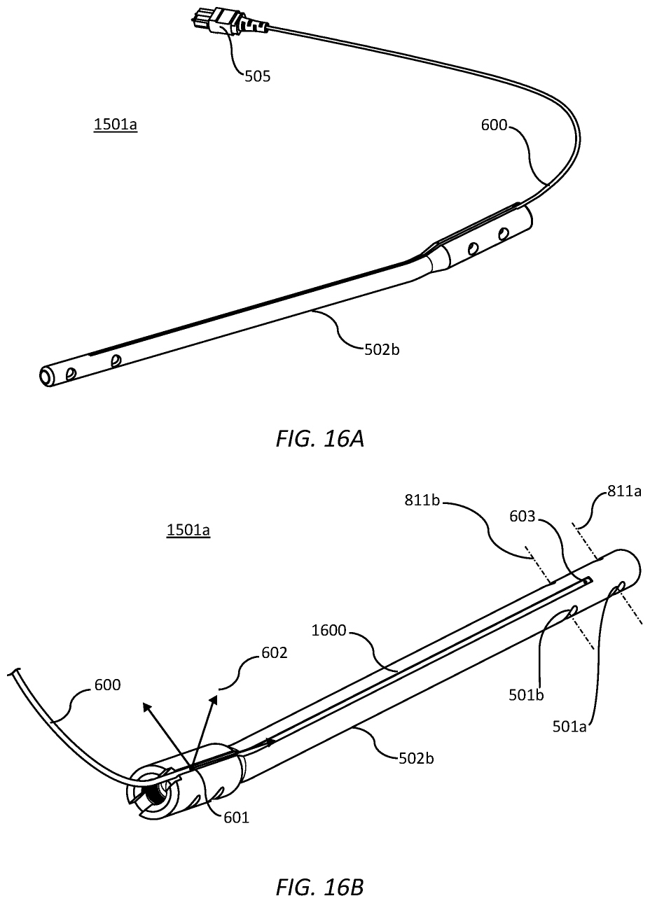

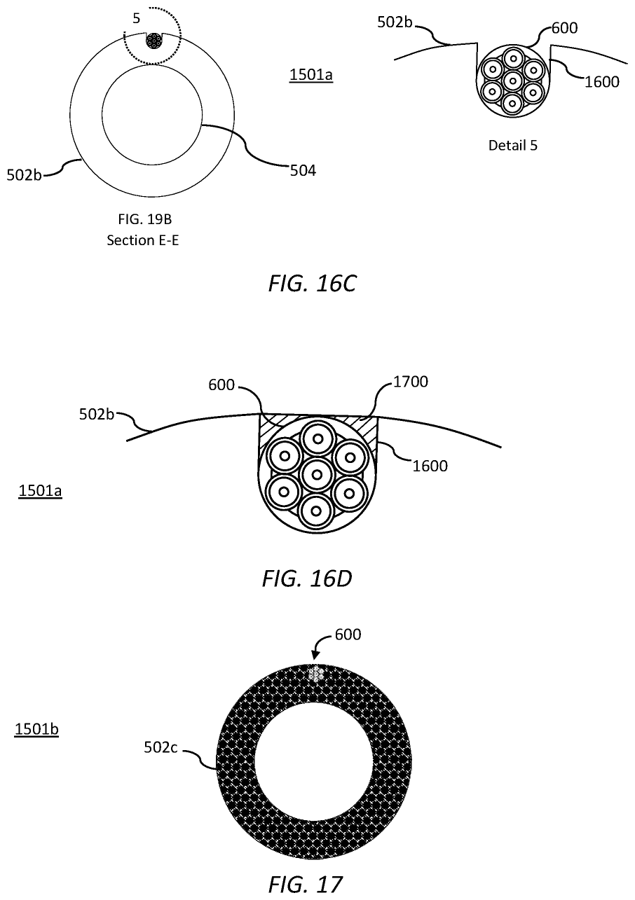

FIGS. 16A-16B are perspective views of shape sensing intramedullary nail 1501a. In this example, an intramedullary nail 502b is integrated with shape sensing cable 600, where the assembly may be packaged sterile to arrive at the user ready for implantation. The first end of shape sensing cable 600 is provided with male multicore connector 505 for operative connection to interrogator 400. A portion of the distal end of shape sensing cable 600 is fixed in a channel 1600 formed in the outer surface of intramedullary nail 502b, bonding base node 601 to a fixed location in rigid communication with the proximal end of intramedullary nail 502b. A portion near the distal end of shape sensing cable 600, including target node 603, is bonded in a fixed location with respect to distal fixation holes 501a and 501b. Shape sensing intramedullary nail 1501a may be provided to the user with calibration information stored on portable memory 1502. In this embodiment, shape sensing cable 600 may be interrogated prior to the surgical procedure, during the manufacturing process for example, to determine the position of target node 603 in base reference frame 602 during the rest condition of intramedullary nail 502b. The positional information may then be saved on portable memory 1502 to be recalled for comparison to the position of target node 603 in the implanted condition where the translation of the distal end of intramedullary nail 502b can be calculated and provided to the user for drill guide adjustment as described heretofore.

FIG. 16C illustrates section and detail views of shape sensing intramedullary nail 1501a, according to the disclosure. In this embodiment, shape sensing cable 600 is rigidly contained in channel 1600 with a fixed fit tolerance during the manufacturing process. Channel 1600 may have depth enabling shape sensing cable 600 to be fully recessed from the outer surface of intramedullary nail 502b. Portions of shape sensing cable 600 extending beyond the outer diameter of intramedullary nail 502b may experience compressive forces between the nail and the inner surface of the canal when implanted, causing strain forces not corresponding to a shape change to be measured and included in the shape reconstruction calculation, which could negatively affect positional accuracy and should be avoided. The portion of shape sensing cable 600 contained in channel 1600 may remain in position during the implantation, or may be removed by the user using a modest axial force after all targeted holes have been drilled. It should be noted that with shape sensing cable 600 bonded to intramedullary nail 502b in this configuration, lumen 504 remains accessible for other instrumentation for the duration of the procedure. In an alternative implementation, shape sensing cable 600 may be further secured in channel 1600 using a suitable implant grade adhesive 1700 as shown in FIG. 16D. Adhesive 1700 may be any thermoset, thermoplastic or ultraviolet cured polymer or other adhesive approved for long-term residence in tissue. After implantation and hole targeting, shape sensing cable 600 may be cut near the proximal end of intramedullary nail 502b and the remnant discarded.

FIG. 17 illustrates a cross section view of a shape sensing intramedullary nail 1501b, according to another embodiment. In this example, an intramedullary nail 502c is manufactured as a composite material consisting of a high-tensile fiber and a thermoplastic, thermoset or ultraviolet-cured polymer matrix where shape sensing cable 600 is embedded in the composite during the layup process.

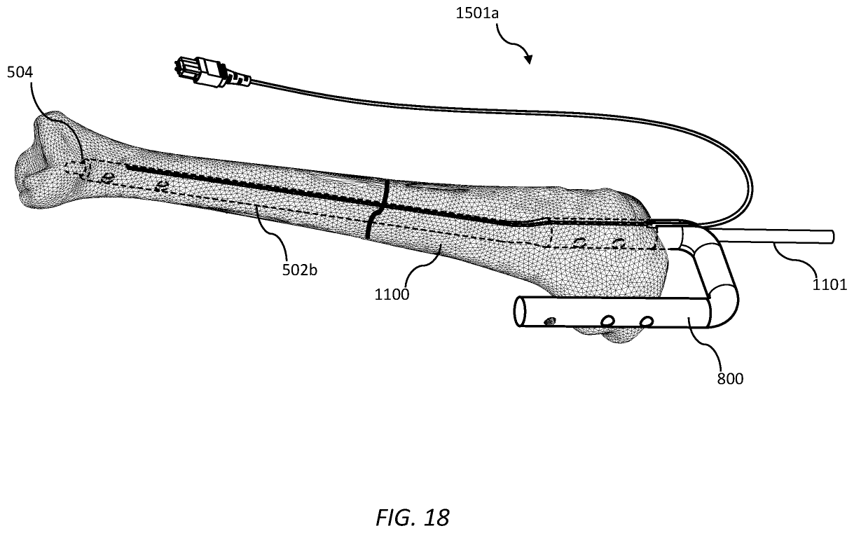

FIG. 18 is a perspective view of shape sensing intramedullary nail 1501a implanted in tibia 1100, according to an embodiment of the present invention. In this example, the intramedullary canal of tibia 1100 is prepared by inserting guidewire 1101 into the canal to span the fracture line, and reaming the canal to provide a slip fit for intramedullary nail 502b. Insertion handle 800 is coupled to the proximal end of intramedullary nail 502b as previously described. Intramedullary nail 502b is then implanted into the prepared canal of tibia 1100 guided by guidewire 1101 passing through lumen 504. Once intramedullary nail 502b is implanted, guidewire 1101 may be removed from lumen 504.

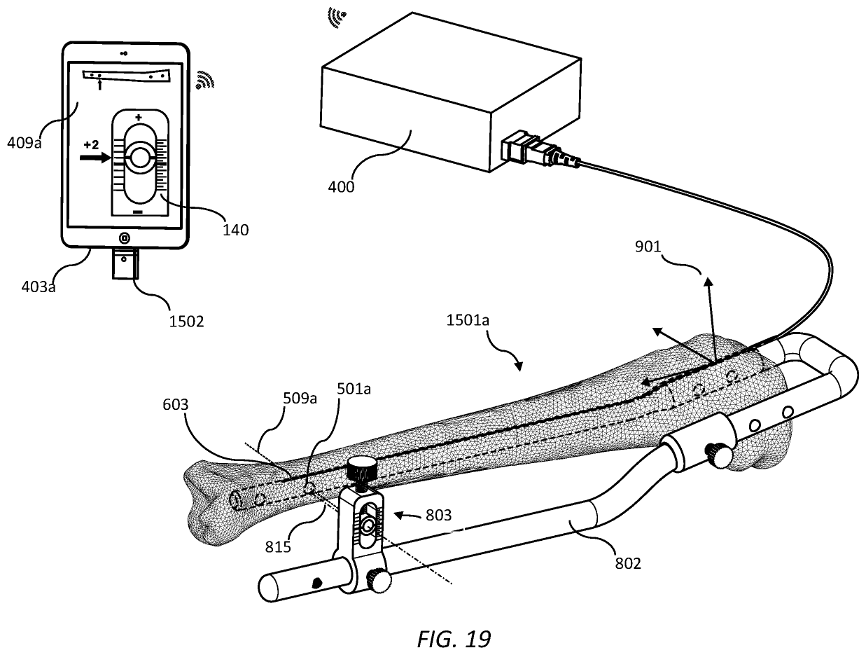

FIG. 19 is a perspective view illustrating targeting system 1500 configured for the targeted drilling of distal fixation hole 501a in shape sensing intramedullary nail 1501a, according to an embodiment of the present invention. After implanting shape sensing intramedullary nail 1501a into the prepared canal of tibia 1100, the user may connect shape sensing cable 600 to interrogator 400 and initialize the system by confirming communication between interrogator 400 and control unit 403a. Calibration data may be uploaded to the targeting software by connecting the provided portable memory 1502 to control unit 403a. In this example, distal fixation hole 514 is selected for targeting, enabling targeting graphic 140 to represent the adjustment of drill guide 813 required to align guide axis 815 to target axis 509a by comparing the second position of target node 603 in secondary reference frame 901 in the implanted shape sensing intramedullary nail 1501a to the first position of target node 603 in the non-implanted shape sensing intramedullary nail 1501a, wherein the first position of target node 603 in the non-flexed is information part of the calibration data provided as a file stored on portable memory 1502. Targeting graphic 140 may be provided to the user on display 409a or additionally on a wearable augmented reality device enabling drill guide 813 and targeting graphic 140 to be in the user's field of view at the same time.

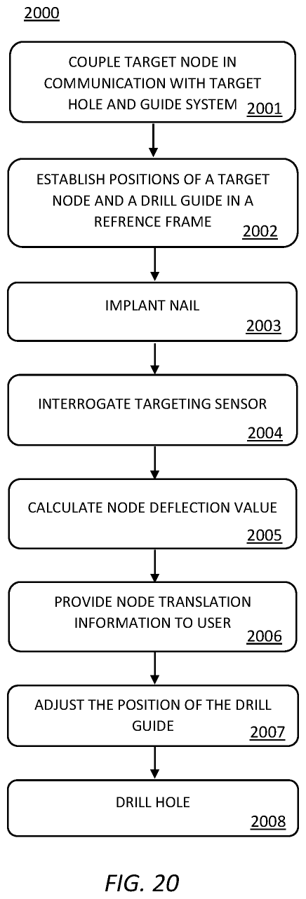

A workflow diagram is presented in schematic form in FIG. 20 to illustrate a method 2000 for aligning a surgical tool to a distal fixation hole in an intramedullary nail, according to an embodiment of the present invention. In step 2001, a guiding system and a targeting sensor, comprising a shape-sensing element, is coupled to an intramedullary nail as described in FIG. 9. In step 2002, a drill guide is positioned in a reference frame at a first location such that its guide axis is aligned to the target axis of the target hole. In one embodiment, the first location of the drill guide may be a known location which to which the user may directly set the drill guide, or in another embodiment, the first location may be established using a guide pin as described in FIGS. 9 and 10. A first position of a target node of the shape sensing element in the reference is established, wherein the target node is placed in rigid communication with the target feature. In one embodiment, the targeting sensor may be placed in the central lumen of the nail and the shape-sensing element interrogated for a first time to determine the first location of the target node as described in FIG. 10. In another embodiment, the shape sensing element may be provided to the user having the target node position information pre-determined to eliminate the calibration step during the surgical procedure, as described in FIG. 15 and FIGS. 16A-16D. In step 2003, the intramedullary nail is implanted into the canal of the fractured bone. Due to variations in anatomical shapes, implant entry position or orientation, or other surgical factors, the distal end of the nail may experience a deflection with respect to the proximal end of the nail which is coupled in rigid communication with the reference frame. In step 2004, the shape-sensing element is interrogated to determine a second position of the target node in the reference frame. In step 2005, the change in position of the target node in the reference frame from the first position to the second position is calculated. In step 2006, information related to the change in position of the target node in the reference frame, which is also related to the change in position of the targeted feature in the reference frame, is provided to the user. In step 2007, the user uses the information related to the positional change of the targeted feature to adjust the position of the drill guide as described in FIGS. 12B-12C. In step 2008, the user may advance a surgical drill through the drill guide to form the fixation hole, as the drill guide is now aligned to the targeted fixation hole in the intramedullary nail.

What is claimed is:

1. A system for targeting a feature on a surgical device, comprising:
a shape sensing element having a first element end and a second element end and a plurality of nodes dispersed therebetween, the first element end coupled to an interrogator, wherein the shape sensing element is operable to receive an interrogation signal from the interrogator and return a modified signal to the interrogator related to the pose of the nodes in a coordinate system, wherein a first node is coupled in communication with a first device end and a second node is coupled in communication with at least one target feature on the surgical device, wherein the surgical device comprises the first device end and a second device end and the at least one target feature located therebetween; and
a data processing system operable to interpret the modified signal to determine the position and orientation of the nodes in the coordinate system, and provide to the user adjustment information related to a change in pose of the at least one feature with respect to the first device end,
wherein a change in pose of the second node with respect to the first node is related to the change in pose of the at least one feature with respect to the first device end,
wherein a guide system is removably coupled to the first device end,
wherein the guide system comprises a guide aperture having a first guide end and a second guide end and a guide axis therebetween, the guide aperture being moveable with respect to the first device end and operable to pass a surgical tool along the guide axis,
wherein the adjustment information is related to a change in pose of the guide aperture with respect to the first device end aligning the guide axis with the at least one target feature.

2. The targeting system of claim 1, wherein the shape sensing element comprises at least one optical fiber, wherein the at least one optical fiber comprises at least one core, wherein the at least one core is operable to conduct the interrogation signal and the modified signal therethrough, wherein the interrogation signal comprises at least one wavelength of light.

3. The targeting system of claim 1, wherein the shape sensing element is removably coupled in communication with the surgical device.

4. The targeting system of claim 1, wherein the shape sensing element is removably coupled to the interrogator.

5. The targeting system of claim 1, further comprising a calibration file containing calibration information relating the pose of the second node with respect to the first node in the coordinate system to the pose of the at least one target feature with respect to the first device end, wherein the calibration file is provided as digital information accessible by the data processing system.

6. The targeting system of claim 1, wherein the surgical device is an intramedullary nail, a fixation plate, a portion of a joint reconstruction implant, a second surgical tool, or any combination thereof.

7. The targeting system of claim 1, wherein information related to the modified signal is provided to the data processing system via a wireless data transmission system.

8. The targeting system of claim 1, wherein the adjustment information is provided to the display via a wireless data transmission system.

9. A medical apparatus, comprising:
a surgical device having a first device end and a second device end and having at least one target feature therebetween; and
a shape sensing element having a first element end and a second element end and a plurality of nodes dispersed therebetween, the first element end connectable to an interrogator, wherein the shape sensing element is operable to receive an interrogation signal from the interrogator and return a modified signal to the interrogator related to the pose of the nodes in a coordinate system;
wherein a first node is coupled in communication with the first device end and a second node is coupled in communication with the at least one target feature,
wherein a guide system is connectable to the first device end and comprises a guide aperture, the guide aperture having a first guide end and a second guide end and a guide axis therebetween, the guide aperture being moveable with respect to the first device end and operable to pass a surgical tool along the guide axis.

10. The medical apparatus of claim 9, wherein the shape sensing element comprises at least one optical fiber, wherein the at least one optical fiber comprises at least one core, wherein the at least one core is operable to transmit an interrogation signal therethrough, wherein the interrogation signal comprises at least one wavelength of light.

11. The medical apparatus of claim 10, wherein at least one core is provided in a helical shape.

12. The medical apparatus of claim 10, wherein the at least one optical fiber includes a plurality of Fiber Bragg Gratings dispersed in the at least one core.

13. The medical apparatus of claim 9, further comprising a calibration file containing calibration information relating the pose of the at least second node with respect to the first node to the pose of the at least one target feature with respect to the first device end, wherein the calibration file is digital information accessible by a data processing system.

14. The medical apparatus of claim 9, wherein the surgical device is an intramedullary nail, a fixation plate, an intramedullary extension of a joint reconstruction implant, a second surgical tool, or any combination thereof.

15. The medical apparatus of claim 9, wherein at least a portion of the shape sensing element is recessed below the outer surface of the surgical device.

16. The medical apparatus of claim 9, wherein the surgical device is constructed from at least two different materials.

17. The targeting system of claim 9, wherein the shape sensing element is removably coupled in communication with the surgical device.

18. A method of aligning a surgical tool to a target feature on a surgical device, comprising the steps:
coupling a first node of a shape sensing element in communication with a first device end of the surgical device, the shape sensing element having a first element end and a second element end and a plurality of nodes therebetween, the first element end coupled to an interrogator, the interrogator operable to provide an interrogation signal to the shape sensing element and receive a modified signal from the shape sensing element related to the position and orientation of nodes in a coordinate system;
coupling a second node of the shape sensing element in communication with at least one target feature on the surgical device;
coupling a guide system to the first device end, the guide system comprising a guide aperture, wherein the guide aperture is moveable with respect to the first device end and comprises a first guide end and a second guide end and a guide axis therebetween, the guide aperture being operable to pass the surgical tool along the guide axis;
providing the interrogation signal to the shape sensing element,
interpreting the modified signal to determine the pose of node in the coordinate system; and
providing adjustment information to the user related to a change in pose of the second node with respect to the first node,
wherein the surgical device comprises the first device end and the second device end and the at least one target feature located therebetween,
wherein a change in pose of the second node with respect to the first node is related to a change in pose of the at least one target feature with respect to the first device end,
wherein the adjustment information is related to a change in pose of the guide aperture with respect to the first device end aligning the guide axis with the at least one target feature.

19. The method of claim 18, wherein the shape sensing element comprises at least one optical fiber, the at least one optical fiber comprising at least one core, wherein the at least one core is operable to transmit the interrogation signal therethrough, wherein the interrogation signal comprises at least one wavelength of light.

20. A method of calibrating a targeting system comprising the steps:
coupling a guide system to a surgical device, the surgical device having a first device end and a second device end and at least one feature therebetween, the guide system comprising a guide aperture, wherein the guide aperture is moveable with respect to the first device end and comprises a first guide end and a second guide end and a guide axis therebetween and operable to pass a surgical tool along the guide axis, wherein the guide system is removably coupled to the first device end;
coupling a shape sensing element to the surgical device, the shape sensing element having a first element end and a second element end and a plurality of nodes therebetween, the first element end coupled to an interrogator, the interrogator operable to provide an interrogation signal to the shape sensing element and receive a modified signal from the shape sensing element related to the position and orientation of the nodes in a coordinate system, wherein a first node is coupled in communication with the first device end and a second node is coupled in communication with the at least one feature;
aligning the guide aperture with the at least one feature;
providing the interrogation signal to the shape sensing element;
interpreting the modified signal to determine the pose of the second node with respect to the first node in the coordinate system; and
registering the pose of the second node with respect to the first node in the coordinate system.

21. The method of claim 20, further comprises including the registered pose of the as a portion of calibration information provided to the user in a calibration file.
